(19) United States
(12) Patent Application Publication
(10) Pub. No.: US 2024/0125952 A1
(43) Pub. Date: Apr. 18, 2024

(54) TIME-OF-FLIGHT POSITRON EMISSION TOMOGRAPHY DETECTOR MODULE

(71) Applicant: The Regents of the University of California, Oakland, CA (US)

(72) Inventor: Gerard Ariño Estrada, Davis, CA (US)

(73) Assignee: The Regents of the University of California, Oakland, CA (US)

(21) Appl. No.: 18/547,533

(22) PCT Filed: Mar. 29, 2022
(86) PCT No.: PCT/US2022/022396

§ 371 Date: Aug. 23, 2023

Related U.S. Application Data
(60) Provisional application No. 63/168,578, filed on Mar. 31, 2021.

(51) Int. Cl. G01T 1/29; A61B 6/03; G01T 1/164; G01T 1/20; G01T 1/22

(52) U.S. Cl. CPC G01T 1/2985; A61B 6/037; G01T 1/1642; G01T 1/20182; G01T 1/22; G01T 1/1644

(57) ABSTRACT

A detector module is provided that can be used as part of a time-of-flight positron emission tomography (TOF-PET) system. The detector module comprises a plurality of emitter elements, each emitter element including an emitter composed of a substance that produces scintillation light and/or Cherenkov radiation in response to gamma photons and, coupled to each of two opposing ends of the emitter, a plurality of photodetectors. The height or thickness of the emitters between their coupled photodetectors is less than 20 mm (e.g., 5-15 mm). The photomultipliers may be silicon photomultipliers or SiPMs that have surface areas less than approximately 9 mm². Due to the quantity of photodetectors, their operating locations at both ends of each emitter, and the relative thinness of the emitters, the emitter elements and the detector module provide a timing resolution better (lower) than 100 ps full width at half maximum.

### RELATED APPLICATION(S)

This application claims the benefit of U.S. Provisional Patent Application Ser. No. 63/168,578 (Attorney Docket No. UC21-676-1PSP), which was filed Mar. 31, 2021 and is incorporated herein by reference.

### BACKGROUND

This disclosure relates to the field of positron emission tomography (PET). More particularly, a gamma photon detector module for use in time-of-flight positron emission tomography (TOF-PET) is provided. The detector module has improved timing resolution and three-dimensional (3D) spatial resolution, and no intrinsic background radiation.

TOF-PET is a leading medical imaging technique that is used extensively for the diagnosis and staging of cancer, coronary diseases, musculoskeletal disorders, and other conditions. The benefits and efficacy of TOF-PET increase as the timing accuracy of the scanner's detectors improve. While existing state-of-the art TOF-PET scanners provide a time accuracy of approximately 210 ps full width at half maximum (FWHM), further improvement is desirable in order to yield image quality that allows diseases and/or other conditions to be diagnosed in very early stages.

### SUMMARY

In some embodiments, a gamma photon detector module for time-of-flight positron emission tomography (TOF-PET) is provided that features greatly improved timing resolution (e.g., less than 100 ps full width half maximum (FWHM)), excellent three-dimensional (3D) position resolution, and no intrinsic background resolution.

In some embodiments, a gamma photon detector module or apparatus comprises multiple emitter elements, wherein each element consists of a material (or metamaterial) that emits scintillation light and/or prompt Cherenkov radiation in response to interaction of the material with gamma photons and, at each of two opposing ends of each emitter, a plurality of photodetectors (e.g., silicon photomultipliers or SiPMs) that detect the light or radiation emitted by the emitter. In some implementations, the emitters are, or include, scintillation crystals, and are less than 20 mm in size from one opposing end to the other.

A detector module may be coupled to a controller (e.g., a computer system, a processor) that receives signals, from the module's emitter elements, that convey information regarding emitter/photon events detected by the elements' photodetectors. In particular, when a pair of gamma photons created by a single electron-positron annihilation is emitted from a subject of a TOF-PET scan along a line of response, and interact with emitters in opposite emitter elements (i.e., one on each side of the subject along the line of response), the emitters' attached photodetectors sense the light and report the event to the controller. Based on the difference in event times reported by the opposite emitters, the controller can identify or estimate the point of origin of the photons, or the annihilation point, within the subject.

In some embodiments, two or more photon detector modules are incorporated into a TOF-PET scanning machine or system, along with a controller and means for communicating between the controller and the emitting elements (e.g., wired and/or wireless communication connections). The machine or system may also include a display for displaying a result or output of a scan of a subject.

### DESCRIPTION OF THE FIGURES

FIG. 1 is a block diagram of a photon detector module in accordance with some embodiments.

FIG. 2 is a flowchart demonstrating a method of using a detector module or system, in accordance with some embodiments.

### DETAILED DESCRIPTION

The following description is presented to enable any person skilled in the art to make and use the disclosed embodiments, and is provided in the context of one or more particular applications and their requirements. Various modifications to the disclosed embodiments will be readily apparent to those skilled in the art, and the general principles defined herein may be applied to other embodiments and applications without departing from the scope of those that are disclosed. Thus, the present invention or inventions are not intended to be limited to the embodiments shown, but rather are to be accorded the widest scope consistent with the disclosure.

In some embodiments, a detector module and/or system is provided for use with or within a time-of-flight positron emission tomography (TOF-PET) system or machine. A detector or detection module disclosed herein may be alternatively termed a “radiation detector module,” a “photon detector module,” or a “gamma photon detector module.” In these embodiments, the detector module provides significantly improved timing accuracy and three-dimensional (3D) spatial accuracy compared to existing detectors.

Traditional detector modules employ relatively thick/tall scintillation crystals (e.g., measuring at least 20 mm) and relatively large photodetectors (e.g., at least 3 mm×3 mm), wherein one photodetector is coupled to one end or face of multiple crystals. The size of the traditional photodetectors limits the timing resolution that can be achieved (e.g., to a value greater than 100 ps) due to their inherent capacitance, which limits how fast they can report photon interaction events.

In contrast, in some embodiments of detector modules provided herein, each of a set of multiple relatively thin/short emitters is coupled to multiple smaller photodetectors on two opposing ends of the emitter. For example, illustrative emitters in these embodiments may range in thickness or height from approximately 5 mm to approximately 15 mm. The multiple photodetectors coupled to each end of an emitter may be smaller than 3 mm×3 mm.

Due to the high density of photodetectors in the disclosed modules, and the increased timing resolution, spatial estimation is improved and results of a TOF-PET scan can be more accurate. In particular, by combining or comparing the output signals of photodetectors at each end of a given emitter, the ability to estimate the depth-of-interaction (DOI) when a photon strikes the emitter is greatly enhanced. The process within the crystal illustratively may feature Cherenkov radiation or scintillation processes to help promote improved timing resolutions (e.g., less than approximately 40 ps FWHM).

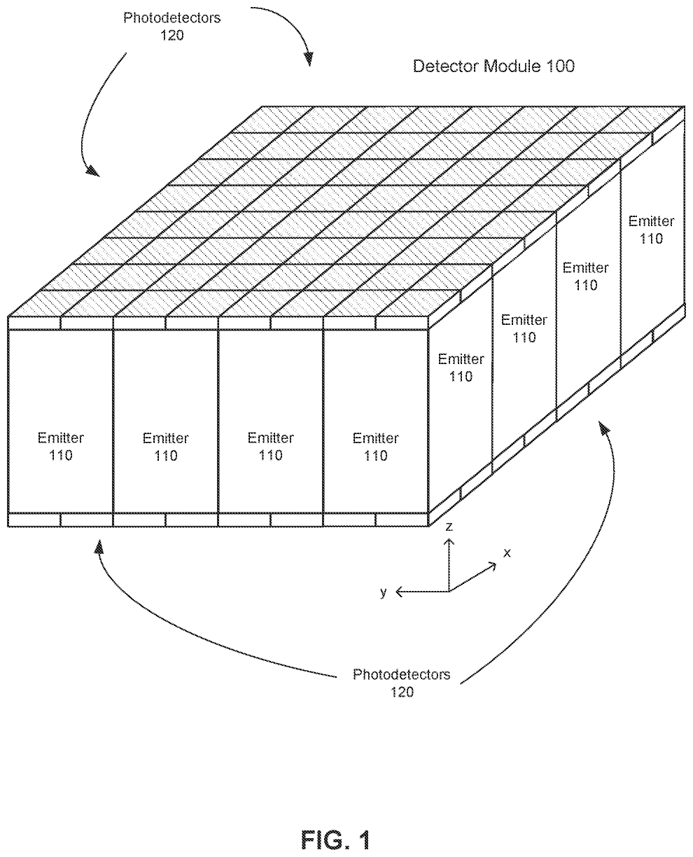

FIG. 1 illustrates a detector module according to some embodiments. In these embodiments, detector module 100 features a set of emitters 110 that are composed of a material or metamaterial that emits scintillation light, Cherenkov radiation, and/or other light when struck by photons. Emitters 110 may be alternatively termed crystals.

Each end of each emitter 110 is connected or coupled to multiple photodetectors 120. In some implementations, photodetectors 120 are silicon photomultipliers (SiPMs). By situating photodetectors at both ends, the capture of light can be improved relative to existing detector modules and the resolution or the ability to identify the source of a gamma photon (or pair of photons) is improved.

In particular, by installing multiple photodetectors at each end of each emitter, spatial resolution in the x- and y-axes (as shown in FIG. 1) is improved. The smaller size of the photodetectors, as compared to existing systems, improves the timing resolution because the smaller photodetectors feature lower capacitance values and can therefore report interactions more rapidly. Spatial resolution in the z-axis is also improved, due at least in part to the smaller dimension of the emitters along that axis.

Module 100 of FIG. 1 features 16 emitters arranged in a 4×4 matrix, but one of ordinary skill in the art will appreciate that a detector module may comprise more (or less) than 16 emitters. The combination of one emitter with its connected photodetectors may be termed an emitter element, while a combination of multiple emitter elements may be termed a detector module or an array of emitter elements. It may be noted that the ratio of photodetectors to emitters in module 100 is greater than 1. In particular, each of the two ends of the detector module exhibits a ratio of 4 (i.e., 64:16) and the entire apparatus features a ratio of 8 (i.e., 128:16). By way of comparison, traditional detector modules feature corresponding ratios less than 1 (e.g., 12:16).

As indicated above, in different embodiments emitters 110 may comprise different materials. For example, the emitters may be composed of a traditional material such as lutetium oxyorthosilicate (LSO). As one alternative, emitters may be made with bismuth germanate (BGO), which emits scintillation light and Cherenkov radiation, emits no background radiation, and may provide the benefit of lower production costs. Other alternatives include thallium chloride (TlCl), thallium bromide (TlBr), and lutetium oxide (Lu₂O₃).

Costs are further reduced because the volume of each emitter 110 is less than the volume of a traditional emitter. In particular, the distance between photodetectors 120 on opposing ends of a given emitter 110 may be less than 20 mm (e.g., 5-15 mm), whereas existing detector modules feature a corresponding dimension of greater than or equal to 20 mm.

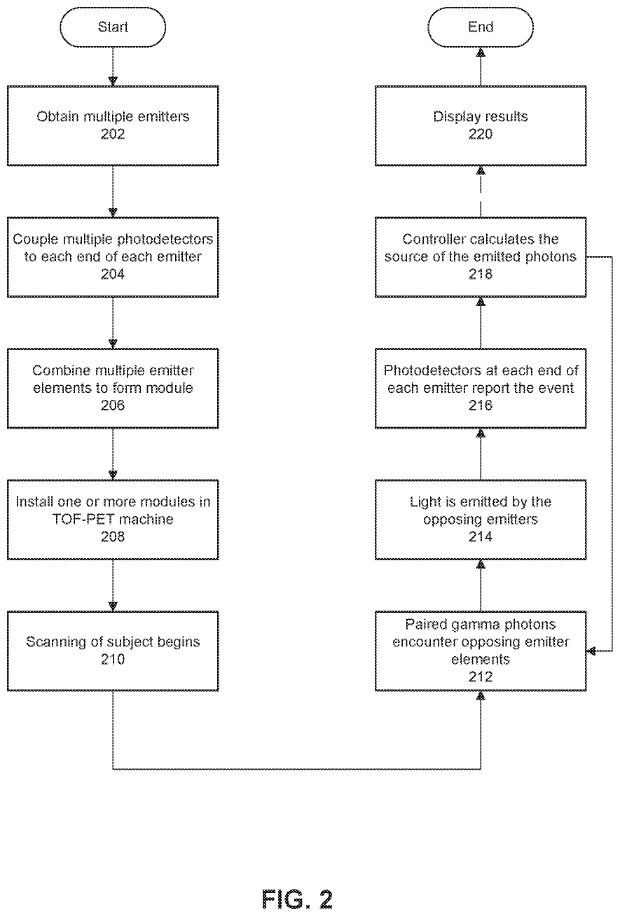

FIG. 2 is a flowchart demonstrating a method of using a detector module or system, according to some embodiments.

In operation 202, multiple three-dimensional (3D) prompt-light emitters are obtained (e.g., via manufacture or assembly). The emitters may feature the same or different dimensions, although in the illustrated embodiments all emitters have at least one dimension of virtually the same length (e.g., along the z-axis depicted in FIG. 1). This dimension may be illustratively termed the “detection dimension” because at each end of this dimension of each emitter, photodetectors will be located to detect light emitted from within the emitter.

In some embodiments, the emitters may be a natural compound comprising a crystal of LSO, BGO, TlCl, etc. In some other embodiments, the emitters may comprise a metamaterial that is a combination of two or more primary materials. In the latter case, the combination may take the form of a heterostructure comprising several macroscopic layers (e.g., alternating layers of approximately 0.1 mm thickness), or a combination at the microscopic level in which two or more components are combined in a way not detectable to the naked eye.

In operation 204, multiple photodetectors (e.g., SiPMs) are attached to each of two opposing ends of each emitter, along the detection dimension, to form emitter elements. As shown in FIG. 1, for example, the photodetectors affixed to a given end of a given emitter may be placed in a matrix (e.g., 2×2, 3×3), although this orientation is not required in all embodiments.

In operation 206, multiple emitter elements are combined to form one detector module, and multiple modules may be assembled. Within each module, the axes of the detection dimension of the emitter elements are aligned in parallel (e.g., with the z-axis in FIG. 1).

In operation 208, multiple detector modules are installed in a positron emission tomography (PET) machine. Specifically, the modules are oriented so that the z-axes of opposite or opposing emitters are perpendicular to the central axis of the scanner. In other words, in a PET machine that scans radially around a cylindrical or circular opening in which the subject is positioned, one or more detector modules are installed at opposing locations such that lines of response mostly coincide with the axes of opposing emitter elements.

In operation 210, a subject (e.g., a human patient) is scanned by the PET machine while a radioactive dye or substance (e.g., a PET tracer) that was injected or inserted into the subject emits gamma photons. The nature of decay of the radioactive substance causes pairs of gamma photons to be emitted simultaneously, in substantially opposite directions (e.g., separated by an angle of approximately 179 to 180 degrees).

In operation 212, paired gamma photons interact with two opposite or opposing emitters (i.e., one emitter in each of two detector modules located opposite each other). Because the photodetectors coupled to the emitters are significantly less dense than the emitters, it is extremely improbable that either gamma photon will interact with a photodetector. Instead, they will pass through the emitters' photodetectors and interact with the connected emitters.

In operation 214, within each of the two emitters, scintillation light and/or Cherenkov light/radiation are released from the point of interaction between the photon and the emitter and propagate toward the photodetectors situated at each end of the emitter element. Each interaction between a gamma photon and an emitter may be termed an event.

In operation 216, at least one photodetector at each end of each of the two emitters detects the light emission and reports the event to a controller (e.g., a computer processor or system coupled to the photodetectors). The information captured by the photodetector(s) and reported to the controller may include any or all of the position/location of the interaction between the photon and the emitter (in 3D), the intensity or energy level of the photon, and a (very precise) time of detection of the interaction.

In operation 218, the controller uses the reported data to calculate the origin of the emitted photons within the subject. In particular, the controller uses the difference in event timestamps reported by the opposing emitters along the line of response to help identify a location within the subject from which the paired photons were emitted. Some events, or the data from some events, may be discarded when they are of low quality or resolution (e.g., because of a low energy level of a photon associated with a particular event, because only one photon from a pair interacted with an emitter).

In some implementations, when photodetectors at both ends of one or both of the opposing emitters detect and report the same event, the controller may first localize the event within the emitter based on the different timestamps assigned to the detection by the photodetectors at opposing ends of the emitter. For example, the controller may average the two timestamps associated with the events reported by the opposing photodetectors of the emitter. Afterward, the controller may compare the values associated with the opposing emitters to isolate the annihilation point within the subject.

An advantage of the design of detector modules described herein is the increase of resolution in locating the annihilation point within the subject along the line of response. More specifically, current TOF-PET scanning systems provide a resolution measured in centimeters, while the described detector modules enable a resolution measured in millimeters.

In operation 220, after repeating operations 212 through 218 multiple times, the controller (or some apparatus operating in conjunction with the controller) displays full or partial results of the TOF-PET scan.

After operation 220, the method ends.

An environment in which one or more embodiments described above are executed may incorporate a general-purpose computer or a special-purpose device such as a hand-held computer or communication device. Some details of such devices (e.g., processor, memory, data storage, display) may be omitted for the sake of clarity. A component such as a processor or memory to which one or more tasks or functions are attributed may be a general component temporarily configured to perform the specified task or function, or may be a specific component manufactured to perform the task or function. The term “processor” as used herein refers to one or more electronic circuits, devices, chips, processing cores and/or other components configured to process data and/or computer program code.

Data structures and program code described in this detailed description are typically stored on a non-transitory computer-readable storage medium, which may be any device or medium that can store code and/or data for use by a computer system. Non-transitory computer-readable storage media include, but are not limited to, volatile memory; non-volatile memory; electrical, magnetic, and optical storage devices such as disk drives, magnetic tape, CDs (compact discs) and DVDs (digital versatile discs or digital video discs), solid-state drives, and/or other non-transitory computer-readable media now known or later developed.

Methods and processes described in the detailed description can be embodied as code and/or data, which may be stored in a non-transitory computer-readable storage medium as described above. When a processor or computer system reads and executes the code and manipulates the data stored on the medium, the processor or computer system performs the methods and processes embodied as code and data structures and stored within the medium.

Furthermore, the methods and processes may be programmed into hardware modules such as, but not limited to, application-specific integrated circuit (ASIC) chips, field-programmable gate arrays (FPGAs), and other programmable-logic devices now known or hereafter developed. When such a hardware module is activated, it performs the methods and processes included within the module.

The foregoing embodiments have been presented for purposes of illustration and description only. They are not intended to be exhaustive or to limit this disclosure to the forms disclosed. Accordingly, many modifications and variations will be apparent to practitioners skilled in the art. The scope is defined by the appended claims, not the preceding disclosure.

What is claimed is:

1. A gamma photon detector apparatus comprising:
multiple emitters comprising material that emits scintillation light and/or Cherenkov radiation in response to interaction with gamma photons; and
a plurality of photodetectors coupled to each of two opposing ends of each emitter.

2. The apparatus of claim 1, wherein a first dimension of each emitter between the two opposing ends is between 5 mm and 15 mm.

3. The apparatus of claim 1, wherein a first dimension of each emitter between the two opposing ends is less than 20 mm.

4. The apparatus of claim 1, wherein the material is bismuth germanate (BGO).

5. The apparatus of claim 1, wherein the material is thallium chloride (TlCl), thallium bromide (TlBr), or lutetium oxide (Lu₂O₃).

6. The apparatus of claim 1, wherein the material is a metamaterial comprising BGO, TlCl, TlBr, and/or Lu₂O₃.

7. The apparatus of claim 1, wherein each photodetector is planar and less than 3 mm×3 mm in size.

8. The apparatus of claim 1, wherein each photodetector is planar and less than 9 mm² in surface area.

9. The apparatus of claim 1, wherein one or more of the multiple photodetectors coupled to each of the two opposing ends of each emitter are silicon photomultipliers (SiPM).

10. The apparatus of claim 1, further comprising one or more photonic crystal layers between each emitter and the plurality of photodetectors coupled to each of the two opposing ends of the emitter.

11. The apparatus of claim 1, wherein the photodetectors provide timing resolution below 100 ps full width at half maximum (FWHM) regarding interaction of gamma photons with the emitters.

12. A time-of-flight positron emission tomography (TOF-PET) system comprising:
at least two gamma photon detector modules, wherein each detector module comprises:
multiple emitters comprising material that emits scintillation light and/or Cherenkov radiation in response to interaction with gamma photons; and
a plurality of photodetectors coupled to each of two opposing ends of each emitter; and
a controller that receives from the photodetectors event data regarding interactions between gamma photons and the emitters.

13. The TOF-PET system of claim 12, further comprising:
a display for displaying results of scanning a subject while the subject emits the gamma photons.

14. The TOF-PET system of claim 12, wherein the controller comprises:
one or more processors; and
memory storing instructions that, when executed by the one or more processors, cause the controller to:
receive event data, from opposing emitters on a line of response, regarding interaction of the emitters with a pair of gamma photons; and
from the event data, calculate a location of emission of the pair of gamma photons.

15. The TOF-PET system of claim 14, wherein the event data provides a timing resolution below 100 ps full width at half maximum (FWHM).

16. A method of using a gamma photon detector module, the method comprising:
installing two or more photon detector modules in a time-of-flight positron emission tomography (TOF-PET) system, wherein each detector module comprises:
multiple emitters comprising material that emits scintillation light and/or Cherenkov radiation in response to interaction with gamma photons; and
a plurality of photodetectors coupled to each of two opposing ends of each emitter; and
receiving, from opposing emitters on a line of response corresponding to a subject of a TOF-PET scan, event data describing interactions between the opposing emitters and a pair of gamma photons; and
from the event data, calculating a location of emission of the pair of gamma photons within the subject.

17. The method of claim 16, further comprising, for each of the two or more photon detector modules:
obtaining the multiple emitters;
assembling multiple emitter elements by coupling, to each of two opposing ends of each emitter, the plurality of photodetectors; and
coupling the multiple emitter elements to form the photon detector module.
